(19) United States
(12) Patent Application Publication
(10) Pub. No.: US 2013/0031030 A1
(43) Pub. Date: Jan. 31, 2013

(54) Urine Sample Collection Apparatus and Method for Chain of Custody Services

(71) Applicants: Ann Marie Carbo, Erie, PA (US); Christopher Carbo, Erie, PA (US)

(72) Inventors: Ann Marie Carbo, Erie, PA (US); Christopher Carbo, Erie, PA (US)

(21) Appl. No.: 13/298,642

(22) Filed: Nov. 17, 2011

Related U.S. Application Data
(60) Provisional application No. 61/512,993, filed on Jul. 29, 2011.

(51) Int. Cl. G06Q 90/00; A61B 5/20

(52) U.S. Cl. 705/500; 600/573

(57) ABSTRACT

An apparatus and method enabling completion of chain of custody services and hands fee collection of urine samples. The technician installs a barrier in a toilet above its water level. Then verifies the donor's identity and directs him to empty his pockets of all contents and remove all accessories from his hands, checks his axillary regions for foreign objects and directs him to wash his hands. The technician installs a collection unit on the toilet seat and directs him to urinate onto the collection unit while under observation at all times. The technician retrieves a urine sample in a sample cup, seals it, and disposes of the used collection unit. The technician directs the donor to sign a record log verifying that he observed the sample collection process and that a new collection unit was used. The technician completes required documentation for the sample and processes it for analysis.

This application take priority from U.S. Provisional Application No. 61/512,993 which is hereby incorporated by reference.

### BACKGROUND

This invention relates to urine specimen collection, especially to the problem of substitution of drug free urine samples for urine that is actually produced by those with illegal drugs in their body.

More and more frequently, urine samples are collected as part of the normal processing for a wide variety of people for a wide variety of reasons. Urine specimen collection for drug screening tests is rife with test subjects who try to cheat the test. Individuals who abuse drugs have a considerable financial and social incentive to tamper with their urine specimens. Depending on the person's circumstances, a positive showing for illegal drugs could mean loss of employment, a change in probation or parole status, being marked as a chronic drug abuser, found to be a government security risk, or being barred or dismissed from the military. Thus, many people have developed very elaborate ways to produce fraudulent urine samples.

Dishonest test subjects have developed a variety of methods to “beat the system” and provide false urine specimens. Technicians who complete chain of custody documents must be vigilant to prevent attempts to cheat. Some test subjects come to the testing facility with urine collected earlier or urine from third parties. Some test subjects will even attempt to conceal these samples in body cavities to simulate that the sample is at body temperature. Some known methods include devices disguised as a male test subject's member that can excrete old drug-free samples of urine out of bags attached to the person's axillary locations as well as balloon-like or container apparatuses located in a female test subject's vaginal cavity which can excrete clean urine upon being adjusted to do so (e.g. a pill container with a seal that is easily punctured by the finger). Some test subjects have been found using synthetic urine alternatives when they cannot find drug free urine.

Dishonest test subjects have also coated their hands with chemicals, powders and adulterants that undermine the results of the urine sample. Such chemicals can be powdered drugs on the fingers, such as Suboxone®, which are easily unnoticed and can be inserted into the urine stream during excretion so they will show up in the urine sample. Bleach is another popular hand coating for a dishonest test subject to use because it contaminates the urine in a way that masks the drug test results. Moreover, new and more elaborate methods are being used on a daily basis.

Therefore, it is important that a collection method be as immune from attacks to their integrity as much as possible. These methods must be cost effective, accurate and not create an undue burden on the test subject.

### SUMMARY

What is presented is an apparatus and methods for collecting urine specimens that prevent the substitution by the donor for a third party and prevent the donor from being able to deposit false urine samples. This apparatus and methods presented are simple, reliable, cost effective and not unduly burdensome ways of reducing, if not eliminating, improper tampering of samples by the test subjects to pass drug screening tests.

The urine sample collection apparatus for chain of custody services requires the use of a standard toilet having a toilet seat that is attached to the standard toilet. The apparatus includes a barrier that is sized to sit in the standard toilet above the water level of the standard toilet and a clean collection unit that is able to cover the toilet seat.

The method disclosed utilizes this apparatus to enable a technician to complete chain of custody services and direct the sample donor to deliver the urine sample in a hands free manner. According to the method disclosed herein, the technician installs the barrier in a standard toilet above the water level in the standard toilet. Then the technician verifies the identity of a donor from which a urine sample is to be collected. The technician directs the donor to empty their pockets of all contents and to remove all non-permanently affixed accessories from the donor's hands and then checks the donor's axillary regions for foreign objects. The technician directs the donor to wash their hands thoroughly. Adequate hand washing removes contaminants that may cause the urine specimen analysis to be unreadable by the laboratory. The technician installs an unused collection unit on the seat of the standard toilet while the donor observes so that the donor may see that the test itself is not compromised. The technician then directs the donor to urinate onto the collection unit so that a sufficient amount of urine collects on the collection unit while the technician observes the donors actions at all times while they are urinating. The technician retrieves a urine sample in a clean unused sample cup, seals the sample cup, and then disposes of the used collection unit. The technician then directs the donor to sign a record log verifying that the donor observed the sample collection process and that a new collection unit had been installed and used. The technician completes the chain of custody documentation for the sealed sample cup and processes the sealed sample cup for urine analysis.

If the donor is male, the sample collection is modified so that the technician instructs the donor to stand in front of the standard toilet and positions the donor so that the donor stands facing sideways to the technician and so that no portion of the donor's back is facing the technician. The technician directs the donor to place one hand behind his back and the other hand to guide urine flow onto the collection unit while having all fingers of the other hand spread. The technician then directs the donor to produce urine onto the collection unit while continuously observing the donor for irregularities in the collection process. It is important the technician is careful to properly make sure that the flow of urine appears to be a completely natural one and nothing suspicious is occurring during the collection process.

If the donor is female, the sample collection is modified so that the technician instructs the donor to refrain from having hands near the donor's vaginal area until the sample collection process is completed and directing the donor to sit on the seat of the toilet that has urine collection unit installed with both hands visibly raised and fingers spread. The technician directs the donor to keep her hands visibly raised and fingers spread until she has completed urinating and steps away from the toilet. As with the male donor, the technician directs the donor to urinate on the collection unit while continuously observing the donor for irregularities in the collection process. When the donor has completed urinating, the technician directs the donor to step away from toilet while continuously observing her behavior and then authorizes the donor, while standing away from the toilet, to wipe the vaginal area with toilet paper until cleansed of urine and then to dispose of the toilet paper into a designated trash receptacle.

Those skilled in the art will realize that this invention is capable of embodiments that are different from those shown and that detail of the devices and methods can be changed in various manners without departing from the scope of this invention. Accordingly, the drawings and descriptions are to be regarded as including such equivalent embodiments as do not depart from the spirit and scope of this invention.

### BRIEF DESCRIPTION OF DRAWINGS

For a more complete understanding and appreciation of this invention, and its many advantages, reference will be made to the following detailed description taken in conjunction with the accompanying drawings.

FIG. 1 shows the apparatus for the urine collection method and installed on a standard toilet;

FIG. 2A shows a perspective view of the collection unit installed on the seat of a standard toilet;

FIG. 2B shows a cross-sectional view of the collection unit installed on the seat of a standard toilet; and

FIG. 3 shows a flowchart of the steps employed in the method according to the present invention.

### DETAILED DESCRIPTION

Referring to the drawings, some of the reference numerals are used to designate the same or corresponding parts through several of the embodiments and figures shown and described. Variations of corresponding parts in form or function that are depicted in the figures are described. It will be understood that variations in the embodiments can generally be interchanged without deviating from the invention.

Urine samples are collected as part of the normal processing for a wide variety of people for a wide variety of reasons. Urine specimen collection for drug analysis is rife with donors who try to cheat the system. Some donors come to the testing facility with urine collected earlier or urine from third parties. Some donors will even attempt to conceal these samples in body cavities to simulate that the sample is at body temperature. Technicians that complete chain of custody documents must be vigilant to prevent attempts to cheat.

There are a many warning signs of cheating. For instance, if the technician observes that the urine sample collected is significantly less than the normal human body temperature, for example at around room temperature, this is an indication that the sample provided is not a fresh sample. Another indication is the behavior of the donor: if shortly prior to the donor's delivery of the urine sample, the donor is seen to be acting in a peculiar manner (i.e. they are seen fiddling in and around their pants or armpits or the donor is insistent in urinating with their back to the person observing their activity) this is probably an indicator that they are planning on attempting to cheat.

To combat these cheating methods, systems of prevention have been patented in the past. For instance a method to ingest a tagging substance was developed in U.S. Pat. No. 5,039,616. Also apparatuses designed to restrictively engage a donor's hands making it impossible to insert a false specimen into a container during a urine test were disclosed in U.S. Pat. No. 6,620,384 and U.S. Pat. No. 6,331,278. These inventions are expensive, not fully reliable, and cause an undue burden on the donor when delivering a urine sample. Moreover, donors may object to ingesting a tagging substance that contains elements unknown to them into their body.

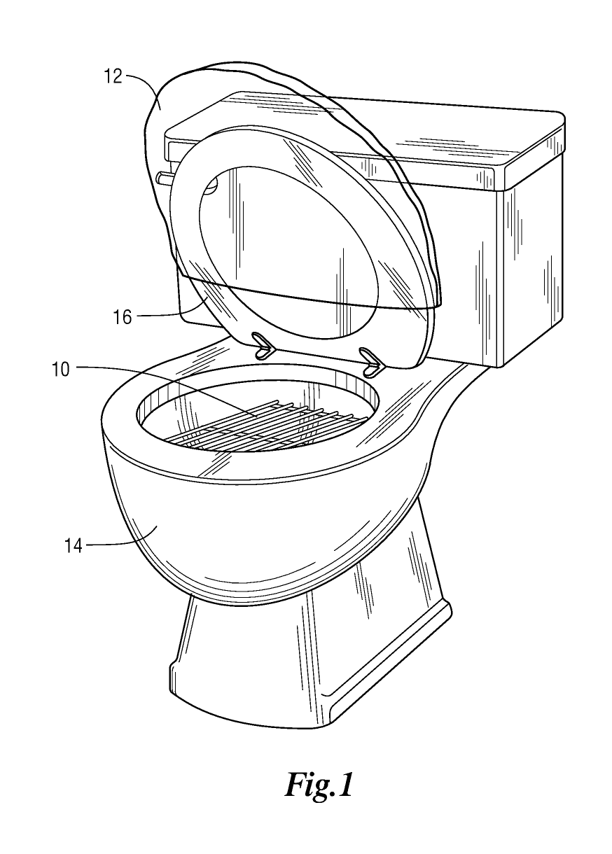

The apparatus and methods disclosed herein for collecting urine specimens attempt to limit the opportunities for cheating as much as possible. Prior to collecting a sample, the technician installs a barrier 10 in a standard toilet 14 as shown in FIG. 1. The barrier 10 is sized to sit in the toilet 14 above the water level of the toilet 14. Generally a barrier 10 that is six inches by ten inches is of sufficient size to suit this purpose. The barrier 10 could be a grill as shown in FIG. 1 or it could be a mesh or other surface that can serve the purpose described herein. The barrier 10 could be a flat surface without openings. Nevertheless the presence of openings allows for any spills and excess urine (after completion of the collection procedure) to pass through the barrier 10 with minimal cleaning required. The barrier 10 could be made of metal, plastic, wood or any other suitably durable material that is easily cleaned. Ideally the material should be reusable so that the barrier 10 can last longer than a year; ultimately making the procedure more cost effective.

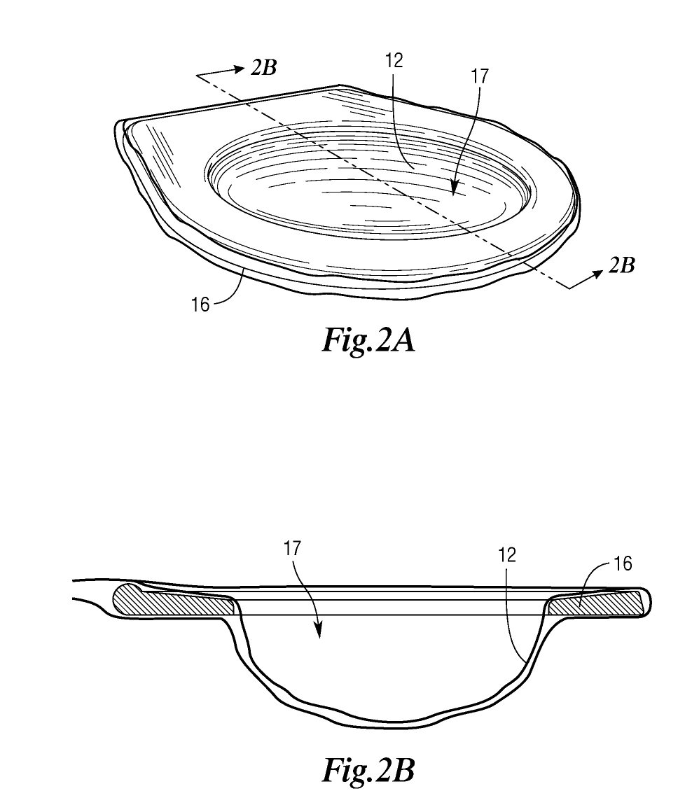

As can be seen in FIGS. 1 through 2B, the toilet seat 16 is covered with a clean collection unit 12 which is usually a disposable plastic bag that is large enough to cover the seat entirely and have enough surface area such that the technician can form a depressed pocket 17 into the toilet seat 16. Preferably, a clear seven to fourteen gallon polyethylene bag has been found to be an acceptable collection unit 12; larger bags may also be used but have been found to be unwieldy and more difficult to handle. Polyethylene bags are easily disposable, cost effective, and sanitary. The bags also serve as a sanitary liner for the toilet seat 16 for women donors while sitting. However, care should be taken to ensure that whatever collection unit 12 is used should come from reputable sources and that the collection unit 12 is not manufactured of or coated with any material which could adversely affect the testing of the collected urine sample. Moreover, the collection unit 12 eliminates the need to hand a specimen cup to the donor which further reduces the risk of tampering with the procedure (e.g. the puncturing of devices within the vaginal cavity by women donors when they are handed a specimen cup, or disguising the flow of false urine as natural by using the specimen cup to visually block the source of the flow, or by the donor adding contaminants or other chemicals that will interfere with the laboratory analysis of the collected urine specimen).

The barrier 10 prevents the collection unit 12 from coming into contact with the water in the toilet 14 so that urine collected in the collection unit 12 is not unduly cooled before it can be collected and allows excess urine to drain into the toilet upon completion. Moreover, the barrier 10 prevents a donor from access to the water in the toilet 14 so they are unable to dilute the specimen with water from the toilet 14. Blocking donors from water in the toilet 14 alleviates the need for dying the water, as required in some prior art procedures, so as to indicate whether any tampering occurred; ultimately reducing the cost of the drug test.

The donor urinates into the depressed pocket 17 formed in the collection unit 12. Soon thereafter, the technician obtains a urine sample directly from the depressed pocket 17 in the collection unit 12 using a clean specimen cup, seals the cup, and disposes the collection unit.

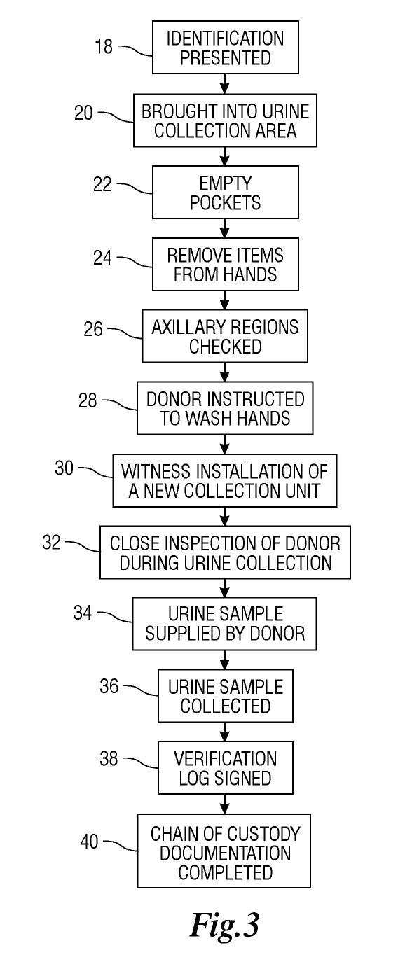

The actual collection chain of custody process involves the technician performing a number of predefined steps to ensure consistent sample collection that becomes routine and as unassailable as possible. FIG. 3 lists these predefined steps that are taken in the novel method. First the donor presents photo identification 18, for the purpose of preventing identity fraud, and any insurance identification required. Once donors are brought into the urine collection area 20, they are not permitted to leave until after the required specimens are obtained otherwise the procedure is voided.

Donors are instructed to empty their pockets 22 of all contents on a designated counter. Purses, coats, scarves, jackets, gloves, etc. are also to be placed on the designated counter. Any items not permanently affixed to the donor's hands 24 such as any jewelry including rings, bracelets, and watches, must be removed and placed on the designated counter. All rings that cannot be removed must be closely examined by the technician and then covered with tape or a glove to prevent their being used to puncture any urine filled bags designed to provide a fake sample. Adhesive bandages or other bandages must be removed from all areas of the donor's hands, fingers, and wrists prior to urine collection to prevent the possibility that such bandages may hide contaminants such as bleach or powdered drugs or sharp objects (again for puncturing urine filled bags or the like). If bandages cannot be removed due to active bleeding, the technician will provide the donor with an unused glove to cover the affected area.

The donor's axillary regions will be checked 26 for foreign objects by the technician. Donors are instructed to wash their hands 28 thoroughly (so as to remove any contaminants) prior to submitting urine samples. The donor will witness 30 the technician obtaining and installing a new unused collection unit 12 on the toilet seat for the donor. It is encouraged that the technician wears medical gloves while installing the collection unit to reduce the risk of cross contamination of the collected urine sample and also to increase the integrity of the collection process. The donor will be observed closely 32 at all times by the technician until the urine specimen has been collected. The technician directs the donor to urinate 34 into the collection unit 12.

Female donors are instructed to refrain from having hands near the donor's vaginal area until the sample collection process is complete. Keeping their hands away from the vaginal area prevents them from puncturing devices that could be located within the vaginal cavity. Female donors will be directed sit on the lined toilet seat 16 with both hands visibly raised and fingers spread until urine delivery is completed. The female donor is directed to urinate into the depressed pocket 17 formed in the collection unit 12. Female donors will be instructed not to have hands near their vaginal areas until they stand a step forward from the toilet 14. The female donor will only be permitted to wipe themselves after they step away from the toilet 14 and will be instructed to dispose the used toilet paper into a designated trash receptacle and not into the toilet 14. At all times, the technician observes the donor for irregularities in the collection process.

Male donors will be directed to stand in front of the standard toilet and positioned so that the donor stands facing sideways to the technician and so that no portion of the donor's back is facing the technician. Male donors will be directed to place one hand behind their back, or raised above the head in plain view, and the other hand to guide urine flow onto the depressed pocket 17 in the collection unit 12 located within the toilet 14. At all times, the technician observes the donor for irregularities in the collection process. It is important the technician is careful to properly make sure that the flow of urine is a completely natural one and nothing suspicious is occurring during the collection process. The technician will collect the urine sample 36 in a clean unused collection cup directly from the collection unit 12. The technician will test the temperature of the urine sample to determine the warmth of the urine with either a thermometer or a temperature strip, either from the sample cup or from within the collection unit 12. The technician then seals the collection cup. The donor will sign a log 38 verifying that they observed that a new collection unit 12 was used and installed on the toilet seat 16, that their urine was collected in a new specimen cup that was sealed and that their collection unit 12 was disposed. The technician completes the chain of custody documentation 40 and processes the urine sample for analysis. If the donor is noncompliant with any of the above steps, the urine sample will be considered void and a repeat attempt to with the proper technique must follow, or the appropriate agency will be notified of the donor's inability to submit a urine sample with the collection guidelines.

This invention has been described with reference to several preferred embodiments. Many modifications and alterations will occur to others upon reading and understanding the preceding specification. It is intended that the invention be construed as including all such alterations and modifications in so far as they come within the scope of the appended claims or the equivalents of these claims.

What is claimed is:

1. A urine sample collection apparatus for chain of custody services that requires the use of a standard toilet having a toilet seat that is attached to the standard toilet comprising:
a barrier that is sized to sit in the standard toilet above the water level of the standard toilet; and
a clean collection unit that is able to cover the toilet seat.

2. The urine sample collection apparatus of claim 1 further comprising said barrier being a grill or a mesh.

3. The urine sample collection apparatus of claim 1 further comprising said barrier being made of metal, plastic or wood.

4. The urine sample collection apparatus of claim 1 further comprising said clean collection unit comprising a disposable plastic bag having enough surface area such that a depressed pocket can be formed into said toilet seat.

5. A method for collecting a urine sample for chain of custody services comprising:
installing a barrier in a standard toilet, the barrier sized to fit in the standard toilet above the water level in the standard toilet;
verifying the identity of a donor from which a urine sample is to be collected;
directing the donor to empty their pockets of all contents and to remove all non-permanently affixed accessories from the donor's hands;
checking the donor's axillary regions for foreign objects;
directing the donor wash their hands;
installing an unused collection unit on the seat of the standard toilet while the donor observes;
directing the donor to urinate onto the collection unit so that a sufficient amount of urine collects on the collection unit;
observing the donors actions at all times while they are urinating;
retrieving a urine sample in a clean unused sample cup;
sealing the sample cup;
disposing of the collection unit;
directing the donor to sign a record log verifying that the donor observed the sample collection process and that a new collection unit had been installed and used;
completing chain of custody documentation for the sealed sample cup; and
processing the sealed sample cup for urine analysis.

6. The method of claim 5 when the donor is male further comprising:
instructing the donor to stand in front of the standard toilet;
positioning the donor so that the donor stands facing sideways to the technician and so that no portion of the donor's back is facing the technician;
directing the donor to place one hand in plain view behind his back or above the head and the other hand to guide urine flow onto the collection unit while having all fingers of the other hand spread; and
directing the donor to produce urine onto the collection unit while continuously observing the donor for irregularities in the collection process.

7. The method of claim 5 when the donor is female further comprising:
instructing the donor to refrain from having hands near the donor's vaginal area until the sample collection process is completed;
directing the donor to sit on the seat of the toilet that has urine collection unit installed with both hands visibly raised and fingers spread;
directing the donor to keep her hands visibly raised and fingers spread until she has urinated and steps away from the toilet;
directing the donor to urinate on the collection unit while continuously observing the donor for irregularities in the collection process;
directing the donor to step away from toilet after urination while continuously observing her behavior; and
authorizing the donor to wipe her vaginal area with toilet paper until cleansed of urine and then to dispose of the toilet paper in the designated trash receptacle.

8. The method of claim 5 further comprising the barrier is a grill or a mesh.

9. The method of claim 5 further comprising the barrier is made of metal, plastic, or wood.

10. The method of claim 5 in which the clean collection unit comprises a disposable plastic bag having enough surface area such that a depressed pocket can be formed into the toilet seat.

11. The method of claim 5 in which the technician tests the temperature of the urine sample provided by the donor.
